(19) United States
(12) Patent Application Publication
(10) Pub. No.: US 2026/0351235 A1
(43) Pub. Date: Nov. 5, 2026

(54) ULTRASOUND LENS HAVING AN INNER LENS AND AN OUTER LENS

(71) Applicant: KONINKLIJKE PHILIPS N.V., EINDHOVEN (NL)

(72) Inventors: Haifeng Wang, State College, PA (US); Ryan M Manning, Lewistown, PA (US)

(21) Appl. No.: 19/488,257

(22) PCT Filed: May 24, 2024
(86) PCT No.: PCT/EP2024/064458

§ 371 Date: Nov. 26, 2025

Related U.S. Application Data
(60) Provisional application No. 63/469,668, filed on May 30, 2023.

(51) Int. Cl. G10K 11/30; A61B 8/00

(52) U.S. Cl. CPC G10K 11/30; A61B 8/00

(57) ABSTRACT

An ultrasound (US) compound lens (206) having an inner lens (306) with a plurality of radii of curvature (ROC's) and an outer lens (304) is disclosed. The compound lens (206) provides a plurality of focal points and a greater depth of focus. The inner lens (306) is disposed adjacent to an acoustic stack (430) of a US transducer (200), and the outer lens (304) is adapted to contact the body (202) being examined.

### BACKGROUND

Ultrasound (US) transducers are ubiquitous in the medical field. US transducers are used in US imaging of practically every aspect of a body. Common to all US transducers are devices to provide azimuthal focus, (along a plane perpendicular to and along the long axis of a transducer probe face) and elevational focus (along a plane perpendicular to and along the short axis of a transducer probe face) to the US waves emitted from an acoustic stack of the US transducer. While azimuthal focusing is provided by beam-steering using beam-forming electronics, elevational focusing is often carried out using a lens.

One aspect of US beam focusing is the depth of focus. Beam-forming electronics normally allow adjustment of the focal point and depth of focus in the azimuthal direction. By contrast, known elevational focusing is done using a fixed lens, such as shown in FIG. 1A, which shows a cross-sectional view of a US transducer 100 from related art. The US transducer 100 comprises an acoustic stack 101 and a US lens 102. The US lens 102 has a constant radius of curvature and a single, substantially fixed, focal point 103. While the US lens is useful in focusing the US waves from the acoustic stack 101, the depth of focus is limited. As such, while comparatively narrow beam is provided as desired to provide acceptable power to effect a desired resolution of the images made by the reflection of the US waves from the point on the body being imaged, before and after the focal point 103, the US beam will diverge. The diverging beam is not well-focused and imaging at locations other than the focal point 103 is compromised.

FIG. 1B shows another US transducer 110 from related art. The US transducer 110 has a compound US lens 104 having an inner lens 105 and an outer lens 106. While there are certain benefits to use of a compound lens 104, the radii of curvature of both the inner lens 105 and the outer lens 106 are constants. As such, the focus from this compound lens is at focal point 103.

Again, the depth of focus of the US transducer 110 is limited. As such, beam divergence before and after the focal point 103 results in undesirably poor image quality at points not at or close to the focal point.

Accordingly, the US transducers from the related art described above have less than desired performance in elevational focusing due to, for example, a limited depth of focus.

What is needed, therefore, is a US transducer that overcomes at least the shortcoming of the US transducers from the related art described above.

### SUMMARY

According to a representative embodiment, an ultrasound (US) compound lens is disclosed. The US compound lens comprises: an outer lens comprising a first material and having a substantially constant radius of curvature across its arc length; and an inner lens comprising a second material that is different from the first material. The inner lens is disposed against the outer lens and comprising a first lens element having a first radius of curvature and a second lens element having a second radius of curvature, which is different from the first radius of curvature. The compound lens provides a first focal point for the first lens element and a second focal point for the second lens element.

According to another representative embodiment, an ultrasound (US) transducer is disclosed. The US transducer comprises: an outer lens comprising a first material and having a substantially constant radius of curvature across its arc length; and an inner lens comprising a second material that is different from the first material. The inner lens is disposed against the outer lens and comprising a first lens element having a first radius of curvature and a second lens element having a second radius of curvature, which is different from the first radius of curvature. The compound lens provides a first focal point for the first lens element and a second focal point for the second lens element. The US transducer further comprises an ultrasound acoustic stack disposed adjacent to the inner lens.

### BRIEF DESCRIPTION OF THE DRAWINGS

The representative embodiments described below are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1A is a cross-sectional view of a known ultrasound transducer used in ultrasound transducers.

FIG. 1B is a cross-sectional view of a known ultrasound transducer used in ultrasound transducers.

FIG. 2 is a perspective view of an ultrasound transducer according to a representative embodiment.

FIG. 3 is a perspective view of an ultrasound lens cap comprising a US compound lens according to a representative embodiment.

FIG. 4 is a cross-sectional view of an ultrasound lens cap comprising a US compound lens according to a representative embodiment.

FIG. 5 is a cross-sectional view of a US compound lens comprising an inner lens and an outer lens and their respective focal points according to a representative embodiment.

FIG. 6 is a cross-sectional view of another US compound lens comprising an inner lens and an outer lens and their respective focal points according to a representative embodiment.

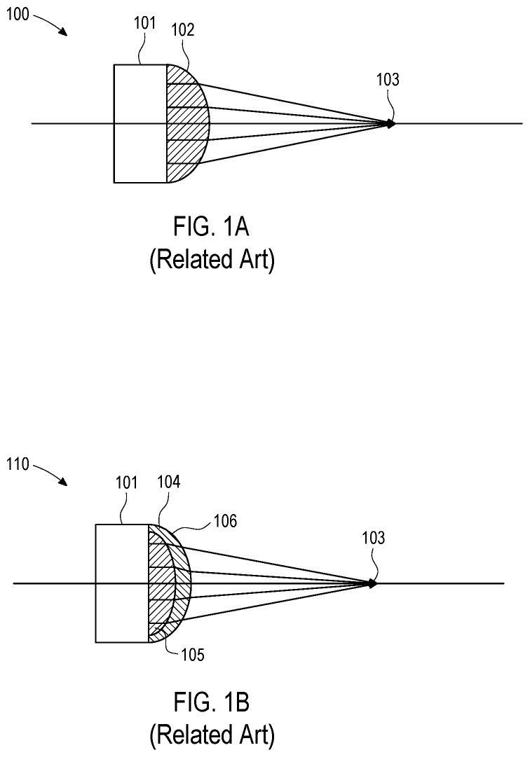

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms “a,” “an” and “the” are intended to include both singular and plural forms, unless the context clearly dictates otherwise. As such, as used herein and unless clearly described as a single device, the term “a device” means “one or more devices,” and the term “the device” means “one or more devices,”

Additionally, the terms “comprises,” and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

As used in the specification and appended claims, the terms “a”, “an” and “the” include both singular and plural referents, unless the context clearly dictates otherwise. Thus, for example, “a device” includes one device and plural devices.

As used in the specification and appended claims, and in addition to their ordinary meanings, the terms “substantial” or “substantially” mean to within acceptable limits or degree. As used in the specification and the appended claims and in addition to its ordinary meaning, the term “approximately” means to within an acceptable limit or amount to one having ordinary skill in the art. For example, “substantially” or “approximately” the same means that one of ordinary skill in the art would consider the items being compared to be the same.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

Generally, the various embodiments described herein relate to US transducers comprising an inner lens and an outer lens. The inner lens is disposed adjacent to the acoustic stack and the outer lens is adapted to contact the surface of a portion of a body to be imaged. The outer lens has a substantially constant radius of curvature, whereas the inner lens has lens elements with differing radii of curvature. The inner lens and the outer lens form a compound lens that provides more than one focal point, and thereby a greater depth of focus for the US transducer than a transducer with a lens with a single focal point. (Notably, as used herein, the depth of focus is as defined by International Electrotechnical Commission (IEC) Standard 6128 (2001) as a distance between two points where the −6 dB beam width is twice of the beam width at the focal point.) As described more fully below, and among other benefits, the compound lens with the inner lens and the outer lens provides improved performance and resolution through increased depth of focus, but avoids the formation of bubbles that cause artifacts in images that plague known US transducers.

Notably, and among other beneficial applications, the US lenses of the transducers of the various representative embodiments are useful in so-called 1.xD (where x is a positive integer) US transducers comprising transducer elements disposed in a center “stripe” and side “stripes” that underlie the US lenses.

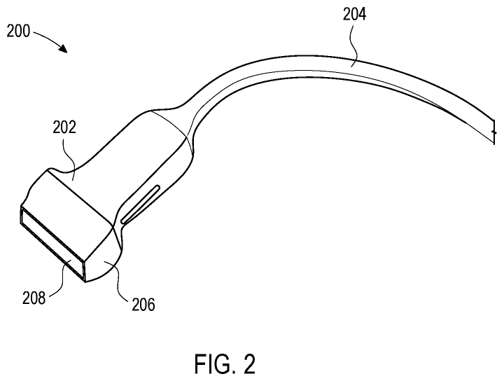

FIG. 2 is a perspective view of a US transducer 200 according to a representative embodiment. The US transducer 200 comprises a body 202 that comprises the various electronics required for its function. These electronics include, but are not limited to an acoustic transducer array comprising a plurality of piezoelectric devices that transmit US waves used for imaging, and receive reflected US waves from the region being imaged. Other electronics include, but are not limited to, beam forming circuitry used to provide azimuthal focusing of the US beams along the long axis of the front face of the US transducer 200, which is illustratively rectangular in shape. The body 202 is connected by a cable 204 to other components (e.g., a display) useful in US imaging.

The US transducer 200 also comprises a compound lens 206. The compound lens 206 comprises a US outer lens (not shown in detail in FIG. 2) and has a rectangular front surface 208 having a long axis along the width of the rectangular front surface 208 and a short axis that is perpendicular to the long axis and is along the height of the rectangular front surface 208. Disposed inside the outer lens and thus not shown in FIG. 2, is an inner lens, which is another component of the US compound lens according to a representative embodiment. As described more fully below, the outer lens has a substantially constant radius of curvature (ROC) across its arc length, and the inner lens comprises a plurality of lens elements. Each of the plurality of lens elements has a different ROC and, accordingly, the compound lens formed by the different lens elements has a plurality of focal points. As will be appreciated, each focal point provides a particular depth of focus, and accordingly, the US transducer 200 provides an increased depth of focus compared to known US transducers.

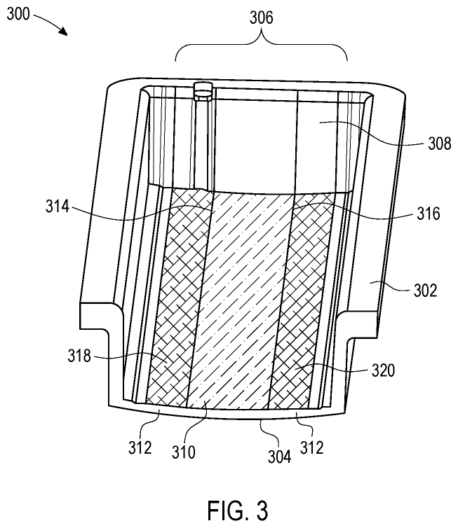

FIG. 3 is a perspective view of a US compound lens 300 according to a representative embodiment. The US compound lens 300 is adapted for attachment to a US transducer such as compound lens 206 shown in and described in connection with FIG. 2 above. Various aspects and details of the US compound lens 300 are common to those described in connection with the representative embodiments of FIG. 2. These common aspects and details may not be repeated in order to avoid obscuring the presently described representative embodiments.

The US compound lens 300 has a body 302 comprising an outer lens 304, which contacts the surface of the body being imaged. As alluded to above, and as described more fully below, the outer lens 304 has a substantially constant ROC across its arc length, which provides one component of compound lenses of various representative embodiments. Beneficially, the outer lens 304 is substantially smooth. By contrast, certain known outer lenses used in some known US transducers have lens elements having different ROC's also have interfaces between the lens elements of the outer lens. These interfaces can cause undesired affects that result in a reduced image quality. Just by way of example, bubbles can form in the matching gels at the interfaces of these lens elements resulting in undesired artifacts and otherwise unacceptable imaging.

The US compound lens 300 also comprises an inner lens 306. The inner lens 306 is disposed against the outer lens 304 on an inner cavity 308 of the US compound lens 300 as shown. The inner lens 306 and the outer lens 304 thus form a compound lens in accordance with a representative embodiment, and useful in US imaging using a US transducer such as US transducer 200. The inner lens 306 comprises a first lens element 310 and a second lens element 312.

As described more fully below, the first lens element 310 has a first radius of curvature and the second lens element 312 has a second radius of curvature, which is different from the first radius of curvature. As a result, and again as described more fully below, the compound lens comprising the inner lens 306 and the outer lens 304 provides a first focal point for the first lens element 310 and a second focal point for the second lens element 312. The multiple lens elements having different ROC's and focal points beneficially provide an increased depth of focus for a US transducer comprising US compound lens 300.

As shown, the first lens element 310 is disposed at a center of the inner lens 306 and comprises one lens element having a first side 314 and a second side 316. The second lens element 312 comprises a first component 318 and a second component 320. As shown in FIG. 3, the first component 318 is disposed adjacent to the first side 314 of first lens element 310 and the second component 320 is disposed on the second side 316 of the first lens element 310.

The inner lens 306 illustratively comprises silicone rubber. The outer lens 304 comprises a material that affords a faster acoustic velocity than the material used for the inner lens 306. Illustratively, the outer lens 304 comprises polyolefin elastomer or polymethylpentene.

As noted above, the inner lens 306 provides two radii of curvature. As described more fully below, the inner lens 306 may comprise more that two lens elements, and thus more than two radii of curvature. For purposes of illustration and not limitation the first lens element 310 has an ROC of 30 mm, and the second lens element 312 has an ROC of 45 mm. The outer lens 304 has an ROC of 35 mm. With these parameters, the focal point for the first lens element 310 is 36 mm, and the focal point of the second lens element 312 is 50 mm. By contrast, a known compound lens having a 30.5 mm inner lens ROC and 35 mm outer lens ROC has a focus length of 42 mm.

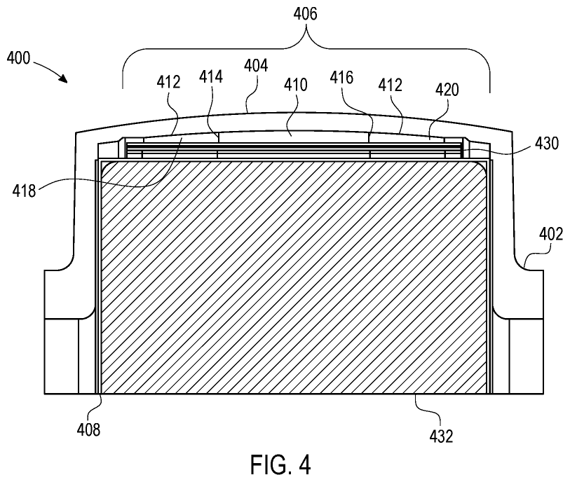

FIG. 4 is a cross-sectional view of a US compound lens 400 according to a representative embodiment. The US compound lens 400 is adapted for inclusion in a US transducer such as ultrasound transducer 200 shown in and described in connection with FIG. 2 above. Various aspects and details of the US compound lens 400 are common to those described in connection with the representative embodiments of FIGS. 2 and 3. These common aspects and details may not be repeated in order to avoid obscuring the presently described representative embodiments.

The US compound lens 400 has a body 402 having an outer lens 404, which contacts the surface of the body being imaged. As alluded to above, the outer lens 404 has a substantially constant ROC across its arc length, which provides one component of compound lenses of various representative embodiments. Beneficially, the outer lens 404 is substantially smooth.

By contrast certain known outer lenses used in US transducers have lens elements having different ROC's. These known outer lenses having lens elements with different ROC's interfaces between the lens elements of the outer lens also have interfaces between the lens elements. These interfaces can cause undesired affects that result in a reduced image quality. Just by way of example, bubbles can form in the matching gels at the interfaces of these lens elements resulting in undesired artifacts and otherwise unacceptable imaging.

The US compound lens 400 also comprises an inner lens 406. The inner lens 406 is disposed against the outer lens 404 on an inner cavity 408 of the US compound lens 400 as shown. The inner lens 406 and the outer lens 404 thus form a compound lens useful in US imaging using a US transducer such as US transducer 200. The inner lens 406 comprises a first lens element 410 and a second lens element 412.

As described more fully below, the first lens element 410 of the inner lens 406 has a first radius of curvature and the second lens element 412 has a second radius of curvature, which is different from the first radius of curvature. As a result, and again as described more fully below, the compound lens comprising the inner lens 406 provides a first focal point for the first lens element 410 and a second focal point having for the second lens element 412. The compound lens with multiple lens elements having different ROC's and focal points beneficially provides an increased depth of focus for a US transducer comprising US compound lens 400 compared to certain known US lenses.

As shown, the first lens element 410 is disposed at a center of the inner lens 406 and comprises one lens element having a first side 414 and a second side 416. The second lens element 412 comprises a first component 418 and a second component 420. As shown in FIG. 4, the first component 418 is disposed adjacent to the first side 414 of first lens element 410 and the second component 420 is disposed on the second side 416 of the first lens element 410.

The US compound lens 400 is further disposed adjacent to an acoustic stack 430. The acoustic stack 430 comprises piezoelectric transducers and electrical circuitry used in the function of a US transducer with which the US compound lens 400 is disposed. The acoustic stack 430 is adjacent to the inner lens 406, which as shown, is adjacent to the outer lens 404.

Electrical connections (not shown) to an electrical cable (e.g., cable 204) are made to connect the acoustic stack 430 to various control and beam forming electronics useful to the function of the US transducer.

Finally, a backing block 432 is disposed in the inner cavity 408 of the US compound lens 400. The backing block 432 is adjacent to the acoustic stack 430 and comprises, among other elements, acoustic matching material used to improve performance of a US transducer comprising US compound lens 400.

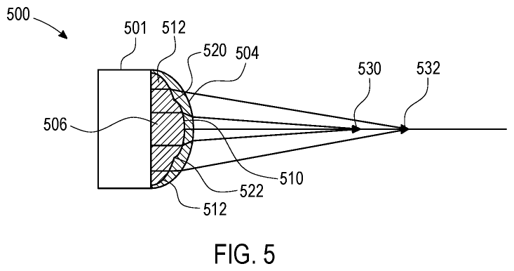

FIG. 5 is a cross-sectional view of a US compound lens 500 comprising an inner lens 506 and an outer lens 504 and their respective focal points according to a representative embodiment. Various aspects and details of the US compound lens 500 are common to those described in connection with the representative embodiments of FIGS. 2-4. These common aspects and details may not be repeated in order to avoid obscuring the presently described representative embodiments.

As shown, the US compound lens 500 is adjacent to a US transducer 501, which is not shown in detail. Notably, the US compound lens 500 is described more fully above in connection with various representative embodiments.

The US compound lens 500 comprises an outer lens 504, which contacts the surface of the body being imaged. As alluded to above, the outer lens 504 has a substantially constant ROC across its arc length, which provides one component of compound lenses of various representative embodiments. Beneficially, the outer lens 504 is substantially smooth. By contrast certain known outer lenses used in US transducers have lens elements having different ROC's. These known outer lenses having lens elements with different ROC's interfaces between the lens elements of the outer lens. These interfaces can cause undesired affects that result in a reduced image quality. Just by way of example, bubbles can form in the matching gels at the interfaces of these lens elements resulting in undesired artifacts and otherwise unacceptable imaging.

The US compound lens 500 also comprises an inner lens 506. The inner lens 506 is disposed against the outer lens 504. The inner lens 506 comprises a first lens element 510 and a second lens element 512.

The first lens element 510 has a first radius of curvature and the second lens element 412 has a second radius of curvature, which is different from the first radius of curvature. As a result, the inner lens 506 provides a first focal point 530 for the first lens element 510 and a second focal point 532 having for the second lens element 512. Specifically, and as will be appreciated by one of ordinary skill in the art, the combination of the first lens element 510 and the outer lens 504 provides the first focal point 530, and the combination of the second lens element 512 and the outer lens 504 provides the second focal point 532. The providing of multiple lens elements having different ROC's and resultant different focal points beneficially results an increased depth of focus for a US transducer comprising US compound lens 400 compared to certain known US lenses.

As shown in FIG. 5, a first interface 520 exists between the first lens element 510 and second lens element 512 on one side, and a second interface 522 exists between the first lens element 510 and second lens element 512 on another side. These interfaces are disposed beneath the outer lens 504, which is substantially smooth. As a result, the first and second interfaces 520, 522 are not in contact with the portion of the body being imaged. Beneficially, and by contrast with certain known US lenses, artifacts and other undesired issues that can arise are substantially avoided by the US compound lens 500 of representative embodiments.

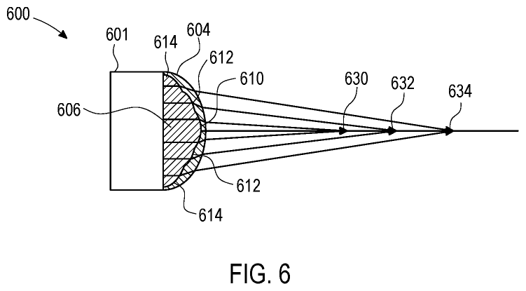

FIG. 6 is a cross-sectional view of a US compound lens 600 comprising an inner lens 606 and an outer lens 604 and their respective focal points according to a representative embodiment. Various aspects and details of the US compound lens 600 are common to those described in connection with the representative embodiments of FIGS. 2-5. These common aspects and details may not be repeated in order to avoid obscuring the presently described representative embodiments.

As shown, the US compound lens 600 is adjacent to a US transducer 601, which is not shown in detail. The US compound lens 600 is described more fully above in connection with various representative embodiments.

The US compound lens 600 comprises an outer lens 604, which contacts the surface of the body being imaged. As alluded to above, the outer lens 604 has a substantially constant ROC across its arc length, which provides one component of a compound lens of various representative embodiments. Beneficially, the outer lens 604 is substantially smooth. By contrast certain known outer lenses used in US transducers have lens elements having different ROC's. These known outer lenses having lens elements with different ROC's interfaces between the lens elements of the outer lens. These interfaces can cause undesired affects that result in a reduced image quality. Just by way of example, bubbles can form in the matching gels at the interfaces of these lens elements resulting in undesired artifacts and otherwise unacceptable imaging.

The US compound lens 600 also comprises an inner lens 606. The inner lens 606 is disposed against the outer lens 604. The inner lens 506 comprises a first lens element 610, a second lens element 612 and a third lens element 614.

The first lens element 610 has a first radius of curvature, the second lens element 612 has a second radius of curvature, and the third lens element 614 has a third radius of curvature. The first ROC, the second ROC and the third ROC are is different from one another. As a result, the inner lens 606 provides a first focal point 630 for the first lens element 610, a second focal point 632 having for the second lens element 612 and a third focal point 634 for the third lens element 614. Specifically, and as will be appreciated by one of ordinary skill in the art, the combination of the first lens element 610 and the outer lens 604 provides the first focal point 630; the combination of the second lens element 612 and the outer lens 604 provides the second focal point 632; and the combination of the third lens element 614 and the outer lens 604 provides the third focal point 634. The providing of multiple lens elements having different ROC's and resultant different focal points beneficially results an increased depth of focus for a US transducer comprising US compound lens 600 compared to certain known US lenses.

It is noted that providing an inner lens having three lens elements and three ROC's is merely illustrative, and providing an inner lens with more that three lens elements is contemplated by the present teachings.

A compound lens for elevational focusing of an US transducer has been described with reference to exemplary embodiments. It is understood that the words that have been used are words of description and illustration, rather than words of limitation. Changes may be made within the purview of the appended claims, as presently stated and as amended, without departing from the scope and spirit of the embodiments. Although estimating and visualizing trajectories of a robotically controlled interventional device on a display has been described with reference to particular means, materials and embodiments, it is not intended to be limited to the particulars disclosed; rather the estimating and visualizing trajectories of a robotically controlled interventional device on a display extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept.

Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. An ultrasound compound lens, comprising:
an outer lens comprising a first material and having a substantially constant radius of curvature across its arc length; and
an inner lens comprising a second material that is different from the first material, the inner lens being disposed against the outer lens and comprising a first lens element having a first radius of curvature and a second lens element having a second radius of curvature, which is different from the first radius of curvature, wherein the US compound lens provides a first focal point for the first lens element and a second focal point for the second lens element.

2. The ultrasound compound lens of claim 1, wherein a depth of focus of the compound lens comprises a sum of a first depth of focus of the first lens element and a second depth of focus of the second lens element.

3. The ultrasound compound lens of claim 1, wherein the first lens element is disposed at a center of the inner lens and comprises one lens element having a first side-and a second side.

4. The ultrasound compound lens of claim 3, wherein the second lens element comprises a first component and a second component.

5. The ultrasound compound lens of claim 4, wherein the first component is disposed adjacent to the first side of the one lens element and the second component is disposed on the second side of the one lens element.

6. The ultrasound compound lens of claim 1, wherein the inner lens further comprises a third lens element having a third radius of curvature which is different from the first radius of curvature and the second radius of curvature, wherein the compound lens provides a third focal point of the third lens element.

7. The ultrasound compound lens of claim 6, wherein a depth of focus of the compound lens comprises a sum of a first depth of focus of the first lens element, a second depth of focus of the second lens element, and a third depth of focus of the third lens element.

8. The ultrasound compound lens of claim 6, wherein: the first lens element is disposed at a center of the inner lens and comprises one lens element having a first side and a second side the second lens comprises a first component having a third side and a second component comprising a fourth side; and the third lens element comprises a third component and a fourth component.

9. The ultrasound compound lens of claim 8, wherein: the first component is disposed adjacent to the first side of the one lens element; the second component is disposed on the second side of the one lens element; the third component is disposed on third side; and the fourth component is disposed on the fourth side.

10. The ultrasound compound lens of claim 9, wherein a depth of focus of the compound lens comprises a sum of a first depth of focus of the first lens element, a second depth of focus of the second lens element, and a third depth of focus of the third lens element.

11. An ultrasound transducer, comprising:
an outer lens comprising a first material and having a substantially constant radius of curvature across its arc length; and an inner lens comprising a second material that is different from the first material, the inner lens being disposed against the outer lens and comprising a first lens element having a first radius of curvature and a second lens element having a second radius of curvature, which is different from the first radius of curvature, wherein the compound lens provides a first focal point for the first lens element and a second focal point for the second lens element; and
an ultrasound acoustic stack disposed adjacent to the inner lens.

12. The ultrasound transducer of claim 11, wherein a depth of focus of the compound lens comprises a sum of a first depth of focus of the first lens element and a second depth of focus of the second lens element.

13. The ultrasound transducer of claim 11, wherein the first lens element is disposed at a center of the inner lens and comprises one lens element having a first side and a second side.

14. The ultrasound transducer of claim 13, wherein the second lens element comprises a first component and a second component.

15. The ultrasound transducer of claim 14, wherein the first component is disposed adjacent to the first side of the one lens element and the second component is disposed on the second side of the one lens element.

16. The ultrasound transducer of claim 11, wherein the inner lens further comprises a third lens element having a third radius of curvature which is different from the first radius of curvature and the second radius of curvature, wherein the compound lens provides a third focal point of the third lens element.

17. The ultrasound transducer of claim 16, wherein a depth of focus of the compound lens comprises a sum of a first depth of focus of the first lens element, a second depth of focus of the second lens element and a third depth of focus of the third lens element.

18. The ultrasound transducer of claim 16, wherein: the first lens element is disposed at a center of the inner lens and comprises one lens element having a first side and a second side the second lens comprises a first component having a third side and a second component comprising a fourth side; and the third lens element comprises a third component and a fourth component.

19. The ultrasound transducer of claim 18, wherein: the first component is disposed adjacent to the first side of the one lens element; the second component is disposed on the second side of the one lens element; the third component is disposed on third side; and the fourth component is disposed on the fourth side.

20. The ultrasound transducer of claim 19, wherein a depth of focus of the compound lens comprises a sum of a first depth of focus of the first lens element, a second depth of focus of the second lens element, and a third depth of focus of the third lens element.
